(19) United States
(12) Patent Application Publication
(10) Pub. No.: US 2011/0077730 A1
(43) Pub. Date: Mar. 31, 2011

(54) BIFURCATED BALLOON STENT

(76) Inventor: Michael S. Fenster, Tifton, GA (US)

(21) Appl. No.: 12/570,151

(22) Filed: Sep. 30, 2009

(51) Int. Cl. A61F 2/06

(52) U.S. Cl. 623/1.3; 623/1.35; 623/1.23

(57) ABSTRACT

A bifurcated stent assembly having a main branch balloon incorporating a sheath associated with the outer wall of the balloon, the sheath having a frangible or separable portion which allows for a guide wire to be passed through the sheath and to be separated from the sheath and the balloon when desired. When the balloon is expanded, the top and bottom edges of the sheath which are associated with the balloon are forced away from each other and cause the frangible or separable portion to separate, allowing the guide wire to be separated from the sheath and the balloon.

### FIELD

The present invention relates to the field of implantable stents, more particularly, to the field of bifurcated stents and methods of using same.

### BACKGROUND

Implanting a stent or performing PTCA on a bifurcated lumen poses challenges for physicians beyond normal stenting procedures to treat stenosis of a blood vessel lumen. Current bifurcated stent designs have the side branch stent separate from the main branch stent, resulting in more time and difficulty than desirable required to install. It would be desirable to have a bifurcated stent design, and method of installation, that would enable the physician to implant both the main branch stent and side branch stent in less time, greater ease and with improved efficiency.

### SUMMARY

Generally described, the present disclosure provides a stent on a novel delivery balloon which allows one to maintain access to a branch vessel guide wire during the stenting process. In a first aspect disclosed is a bifurcated stent assembly comprising a main branch stent unit comprising a main branch tubular stent, a main branch expandable member at least partially disposed within the stent material and having an outer wall, an inner wall, a proximal end, a distal end, an expandable portion disposed between the proximal and the distal ends, and a sheath associated with the outer wall of the expandable portion and being sized to allow a guide wire to pass therethrough. The sheath comprises a wall having an inner surface and an outer surface, a proximal opening, a distal opening, a top portion associated with the outer wall of the expandable member, a bottom portion generally parallel to and proximate to the top portion and associated with the outer wall of the expandable member, a frangible portion disposed between the proximal and distal openings and between the top and bottom portions, the frangible portion having a closed first configuration having a generally tubular shape when the main branch expandable member is in an uninflated configuration and an open second configuration when the main branch expandable member is in an inflated configuration whereby the frangible portion is separated and the top and bottom portions are separated so as to define a sheath edge opening therethrough, allowing a guide wire which is disposed within the sheath to be removable from the sheath via the sheath edge opening. The stent assembly also comprises a side branch stent unit comprising a side branch tubular stent, a side branch expandable member at least partially disposed within the stent material and having a proximal end, a distal end, and, an expandable portion disposed between the proximal and the distal ends.

The sheath frangible portion may be configured in any of several possible configurations, including, but not limited to, a series of perforations, zipper-like interleaved tabs and recesses, overlapping top and bottom edges, and the like. When the main branch expandable member is inflated the frangible portion separates to create an opening so that portion of the side branch guide wire disposed within the sheath can be released from the sheath via the opening.

Another aspect of the present disclosure further provides a method for treating a bifurcated vessel having a main branch and a side branch using a bifurcated stent assembly as described herein, comprising inserting the branch stent in the branch vessel; inserting the main vessel balloon in the main vessel; inflating the main vessel balloon; pulling back on the branch vessel stent until resistance is felt; deploying the branch vessel stent; removing the branch vessel stent balloon; inflating the main vessel balloon; removing the branch vessel stent; inserting the stent assembly over both the main vessel guide wire and the branch vessel guide wire; inserting the stent assembly into the main vessel; advancing and deploying the stent assembly in the main vessel; advancing the branch vessel balloon over the branch vessel guide wire; and, inflating the main vessel balloon and the branch vessel balloon.

Other features of the present disclosure will become apparent upon reading the following detailed description of embodiments, when taken in conjunction with the appended claims.

### BRIEF DESCRIPTION OF THE DRAWINGS

The invention is illustrated in the drawings in which like reference characters designate the same or similar parts throughout the figures of which:

FIG. 1A is a side view in cutaway of a bifurcated blood vessel lumen and a bifurcated stent according to one exemplary embodiment of the present invention showing a main branch stent unit installed and the main stent balloon expanded.

FIG. 1B is a schematic side view of one exemplary embodiment of a bifurcated stent assembly according to FIG. 1A.

FIG. 2 is a schematic view of a detail of a first exemplary embodiment of a frangible sheath associated with a main branch balloon.

FIG. 3 is a schematic view of a detail of a second exemplary embodiment of a frangible sheath associated with a main branch balloon.

FIG. 4 is a schematic view of a detail of a third exemplary embodiment of a frangible sheath associated with a main branch balloon.

FIG. 5 is a schematic view of a detail of a fourth exemplary embodiment of a frangible sheath associated with a main branch balloon.

FIG. 6A is a schematic view of a detail of a fifth exemplary embodiment of a frangible sheath associated with a main branch balloon.

FIG. 6B is a side elevation schematic view of the embodiment of FIG. 6A.

FIG. 7 is a schematic view of a detail of a sixth exemplary embodiment of a frangible sheath associated with a main branch balloon.

FIG. 8 is a schematic view of a detail of a seventh exemplary embodiment of a frangible sheath associated with a main branch balloon.

FIG. 9 is a schematic view of a detail of a portion of a stent showing a balloon in an uninflated configuration and the frangible portion of the sheath intact.

FIG. 10 is a schematic view of a detail of a portion of a stent showing a balloon in an inflated configuration and the frangible portion of the sheath separated.

FIG. 11 is a side cutaway view of the detail view shown in FIG. 9.

FIG. 12 is a side cutaway view of the detail view shown in FIG. 10.

FIG. 13A is a schematic view of a bifurcated stent according to one exemplary embodiment implanted in both lumens of a bifurcated vessel.

FIG. 13B is a detail of FIG. 13A showing a main vessel balloon (without the stent, to better show the construction) with a sheath, the dashed lines showing the main vessel guide wire passing through the main branch balloon area and the side branch guide wire passing through the sheath.

FIG. 14 is a schematic view of a bifurcated lumen with a main branch guide wire and a side branch guide wire inserted, illustrating part of one exemplary embodiment of a first method of implanting a bifurcated stent assembly of the present disclosure.

FIG. 15 is a schematic view according to the method being described for FIG. 14 and showing a side branch stent unit in an initial undeployed or collapsed position.

FIG. 16 is a schematic view according to method being described for FIG. 15 and showing insertion of a main branch balloon.

FIG. 17 is a schematic view according to method being described for FIG. 16 and showing the side branch stent in position with the main branch balloon inflated.

FIG. 18 is a schematic view according to method being described for FIG. 17 and showing the side branch balloon inflated and the stent deployed.

FIG. 19 is a schematic view according to method being described for FIG. 18 and showing the side branch stent deployed and both balloons removed.

FIG. 20 is a schematic view according to method being described for FIG. 19 and showing the side branch stent deployed and the main branch stent in position for deployment.

FIG. 21 is a schematic view according to method being described for FIG. 20 and showing both stents deployed.

FIG. 22 is a schematic view according to method being described for FIG. 21 and showing an additional balloon placed on the side branch wire through the main branch stent.

FIG. 23 is a schematic view according to method being described for FIG. 22 and showing simultaneous inflation of both balloons.

FIG. 24 is a schematic view according to a second method of implanting a bifurcated stent of the present disclosure and showing a main branch stent in position and deployed.

FIG. 25 is a schematic view according to the method of Claim 24 and showing the main branch stent in position and the side branch balloon being placed into the side branch.

FIG. 26 is a schematic view according to the method of Claim 25 and showing the simultaneous inflation of the balloons.

FIG. 27 is a is a schematic view according to the method of Claim 26 and showing the side branch stent deployed through the main branch stent.

FIG. 28 is a schematic view according to the method of Claim 27 and showing both balloons inflated and stents deployed.

### DETAILED DESCRIPTION

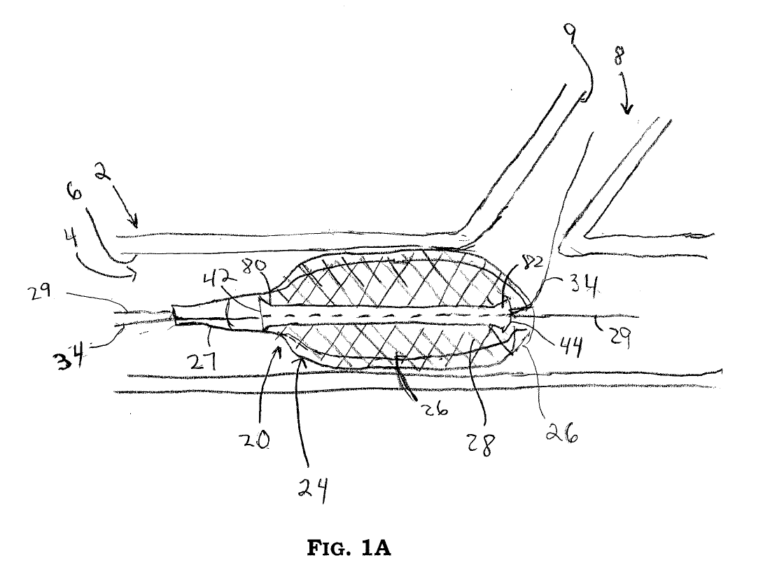

FIGS. 1A and 1B shows a bifurcated vessel 2 having a main lumen 4 defined by an inner wall 6, and a side branch lumen 8 defined by an inner wall 9. In one exemplary embodiment, the present disclosure provides a stent assembly 20 having an expandable main branch stent unit 22 and a side branch stent unit 24. The main branch stent unit 22 has an inflatable main branch balloon 26 or other expandable member which is associated with a main branch catheter 27. The balloon 26 is at least partially disposed within a stent material 28, which may be a mesh, coil, rings, lattice or other expandable material or structure known to those skilled in the art. Typically, the stent material will be generally tubular in configuration. For purposes of the present disclosure, a balloon will be discussed as an illustrative, nonlimiting example of an expandable member. It may be possible to use other expandable members, such as, but not limited to, a tamponading member, such as an umbrella shaped structure, or the like. The purpose of the balloon is to expand the stent from an initial collapsed configuration which enables inserting into the lumen to an expanded or deployed configuration at the intended site in which the stent is to reside. The present invention is also contemplated for use with self-expanding stents. The main branch stent unit 22 also includes a guide wire 29.

The side branch stent unit 24 has an inflatable side branch balloon 30 (as described above) which is at least partially contained within a side branch stent 32 (which may be made of the same material and in the same structural configuration as the main vessel stent 28 or may be made of a different material or have a different structural configuration). The side branch stent unit 24 also includes a side branch vessel guide wire 34 and a side branch catheter 36.

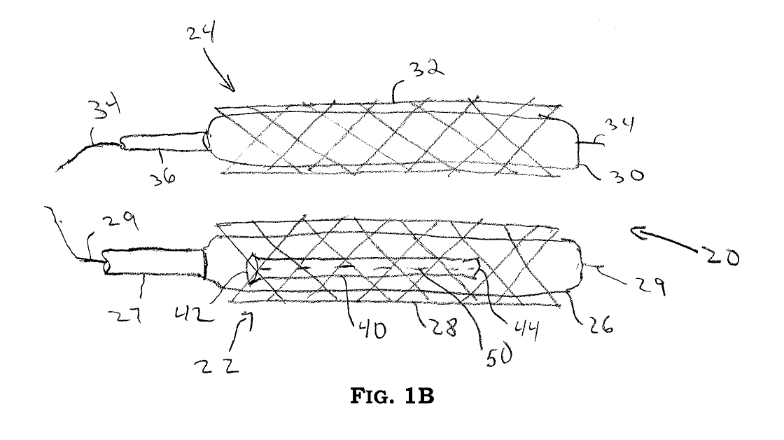

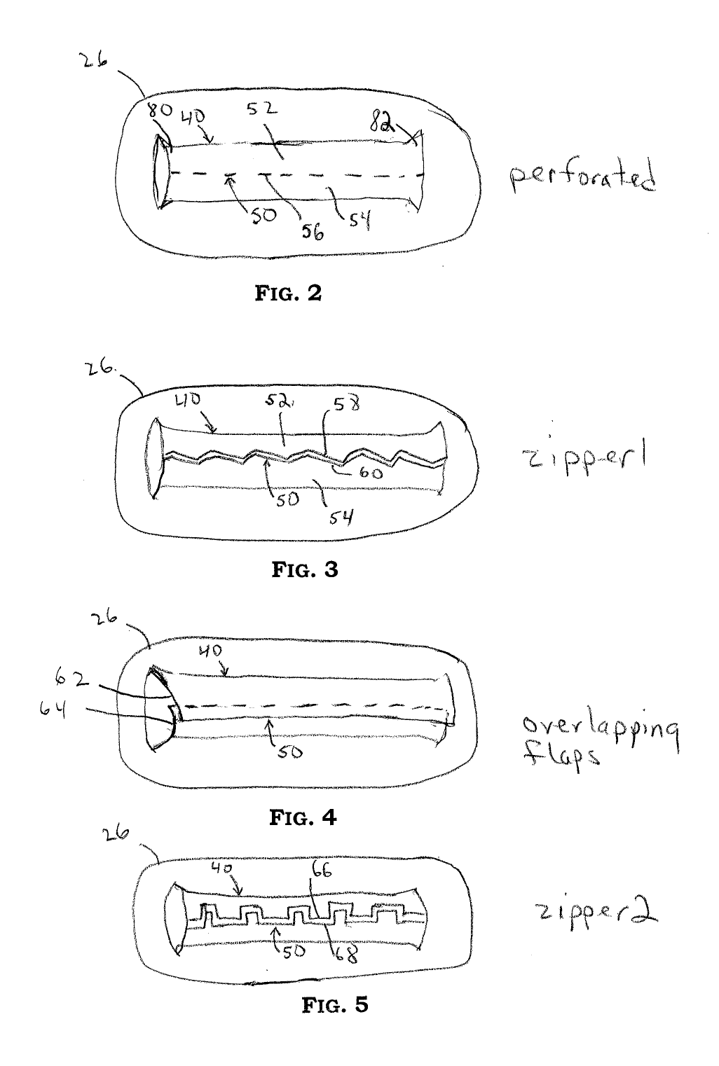

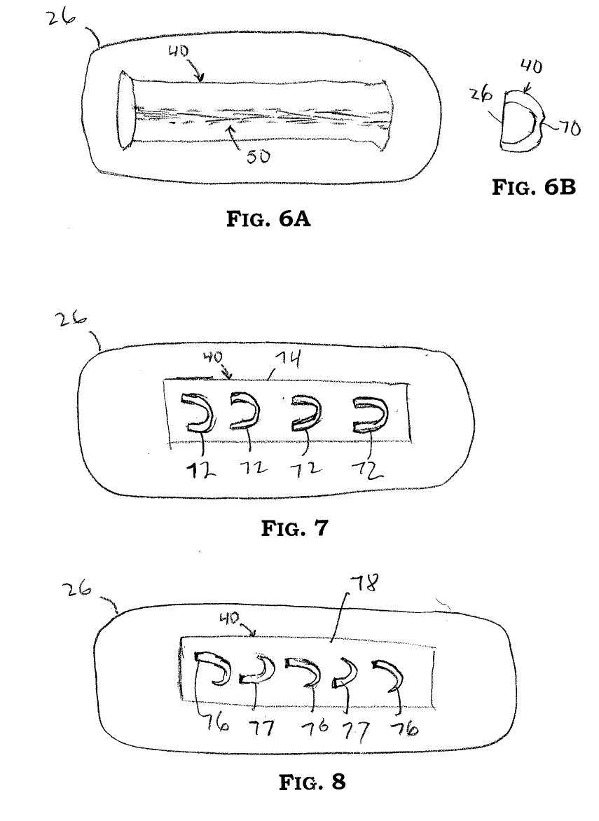

The main branch balloon 26 has a sheath 40 associated with the exterior of the balloon 26. The sheath 40 is elongated, and has a proximal (entrance) end 42 and a distal (exit) end 44. A portion 50 along the length of the sheath 40 is frangible. For the purposes of the present disclosure the term “frangible” means an area which separable, tearable, rupturable or otherwise able to separate generally along the axial line of the sheath to allow a guide wire passing through the sheath to separate from the sheath through the wall of the sheath, rather than withdrawing axially through the end of the sheath. The frangible portion may be constructed in any of several possible configurations, several exemplary embodiments being shown in FIGS. 2-8. The frangible portion may have a top edge 52 and bottom edge 54. FIG. 2 shows the frangible portion 50 as a series of perforations 56. FIG. 3 shows the frangible portion 50 comprising two rows of interlaced teeth 58, 60, similar to a zipper. FIG. 4 shows the sheath 50 as having an axial first flap portion 62 which overlaps a second flap portion 64, whereby the two flap portions can separate from one another. FIG. 5 shows the frangible portion 50 as two rows of teeth 66, 68 in an alternating configuration. FIGS. 6A and 6B show the frangible portion 50 as an area 70 of thinner wall thickness than the rest of the sheath 40. FIG. 7 shows a sheath 40 having at least one and preferably a number of loops 72 attached to a platform 74 which is attached to the balloon 26, whereby the loops 72 can be separated. FIG. 8 shows a sheath 40 having at least one and preferably a number of curved hooks or barbs 76, 77 which alternative directions, the hooks being attached to the balloon directly, or by way of a platform 78. The hooks 76, 77 are somewhat flexible and the branch guide wire 32 can be separated when the hooks 76, 77 flex to release the guide wire 32. It is important that the sheath frangible portion 50, when separated, avoid or minimize the likelihood of stray material separating from the sheath 40 and passing into the bloodstream.

The proximal (entrance) and distal (exit) ends 42, 44 of the sheath 40 may be reinforced at reinforced areas 80, 82 (see FIG. 2), respectively, so that the guide wire does not puncture the balloon 26 when inserted. The reinforced areas 80, 82 are still separable or frangible and separates at the appropriate time. In one exemplary embodiment, the reinforced area 80, 82 can be the same material as the rest of the sheath, and having an increased thickness. Alternatively, the reinforced area 80 or 82 can be made of a different material from the rest of the sheath. In one embodiment, the distal end 44 of the sheath 40 is reinforced. In an alternative embodiment, both the proximal end 80 and distal end 82 of the sheath are reinforced.

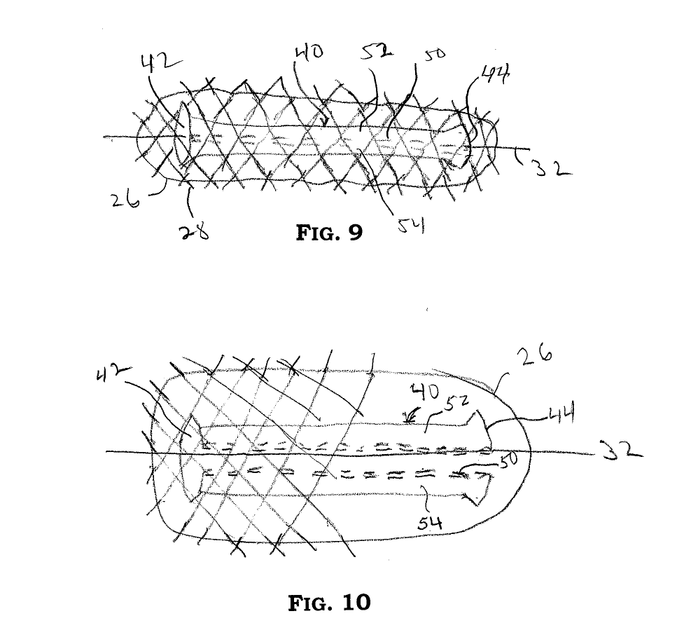

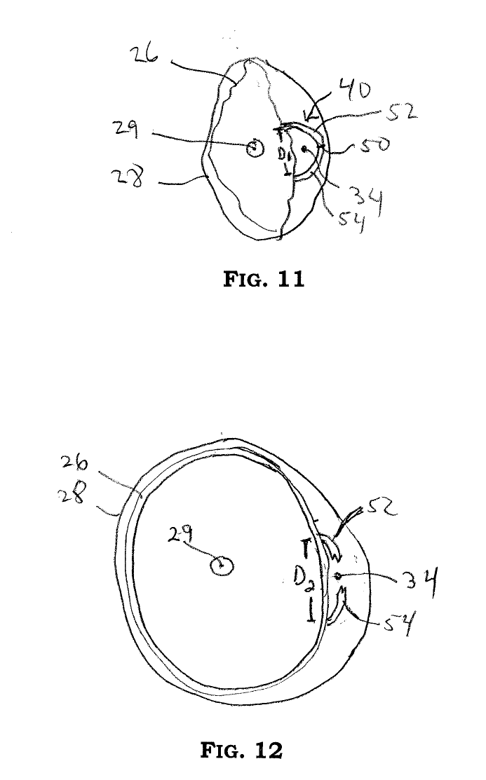

As shown in FIGS. 9-12, the sheath 40 has a top edge 52 and a bottom edge 54, both edges being attached to the balloon 26. When the balloon 26 is expanded the diameter of the balloon 26 expands from the unexpanded configuration (FIGS. 9, 11) into the expanded configuration (FIGS. 10, 12), the distance between the top edge 52 and bottom edge 53 increases, leading to stress being placed on the frangible portion 50 of the sheath 40. Upon application of sufficient stress, the frangible portion 50 ruptures, tears, separates, parts, un-overlaps, or the like, depending on the embodiment, allowing the side branch guide wire 32 to separate from the balloon 26. The stent 32 is disposed outside and around at least a portion of the sheath 40 and balloon 26.

The sheath 40 of the present invention allows for both guide wires 26, 32 to be simultaneously inserted in the stent assembly 20 (the main vessel guide wire 26 being inserted into the stent proximal opening and the branch vessel guide wire 32 being inserted into sheath proximal end 42 opening). The stent assembly 20 with the main vessel balloon 26 is inserted into the main branch lumen 4.

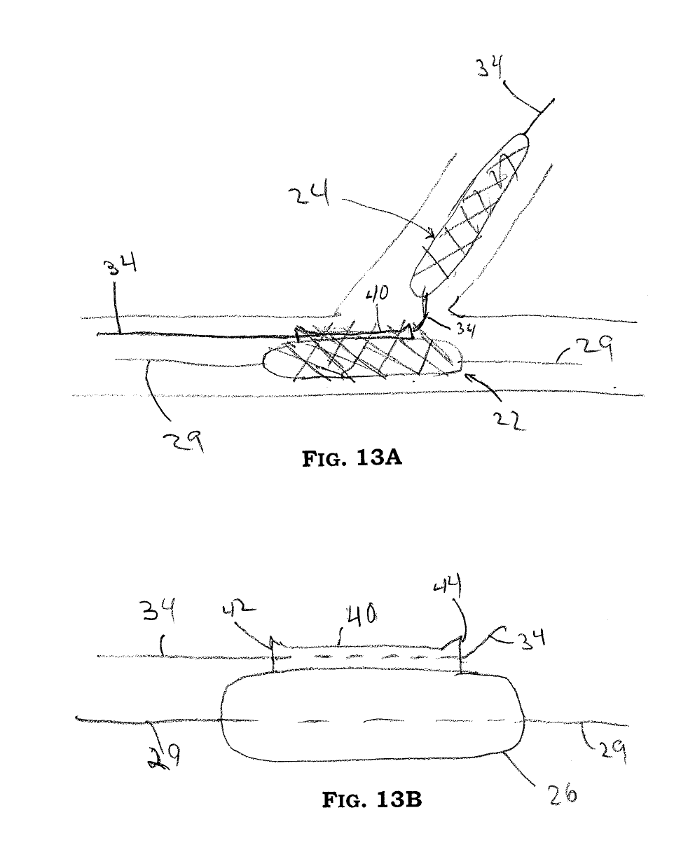

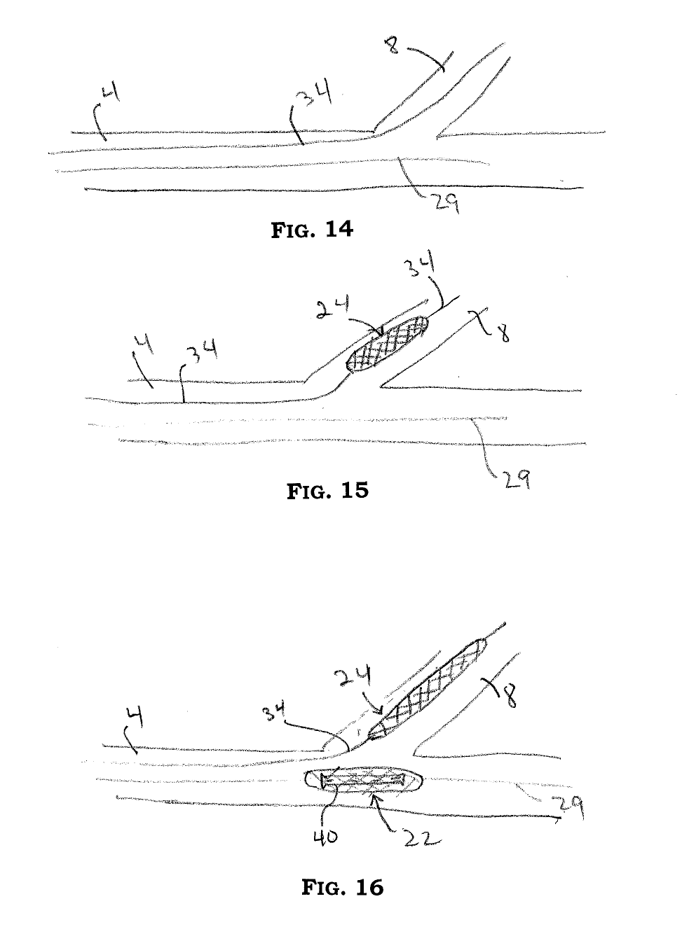

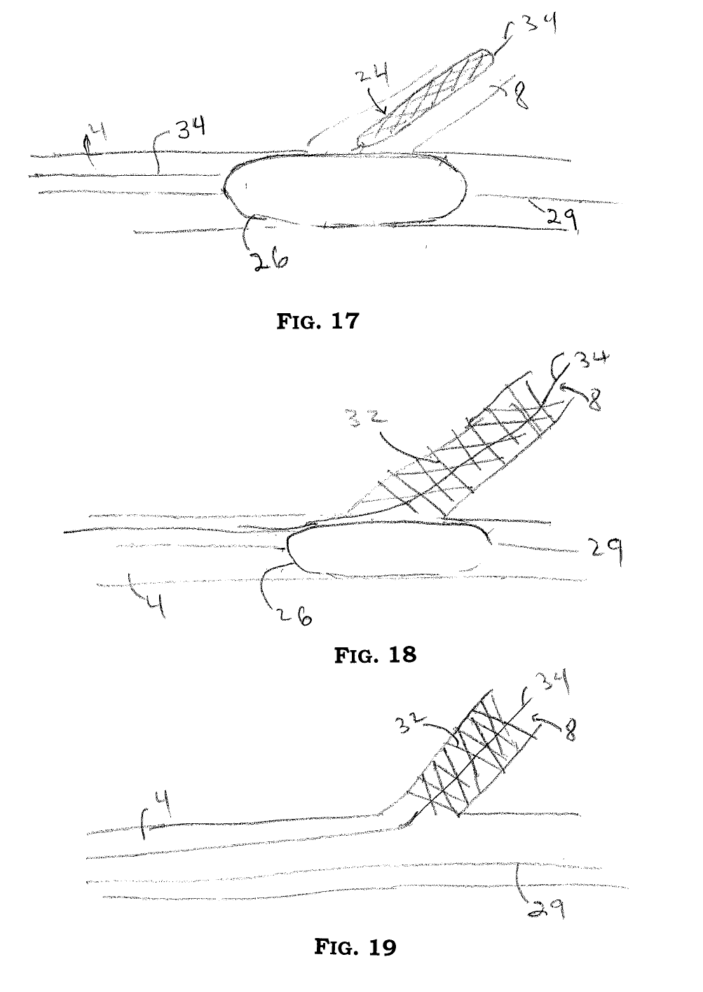

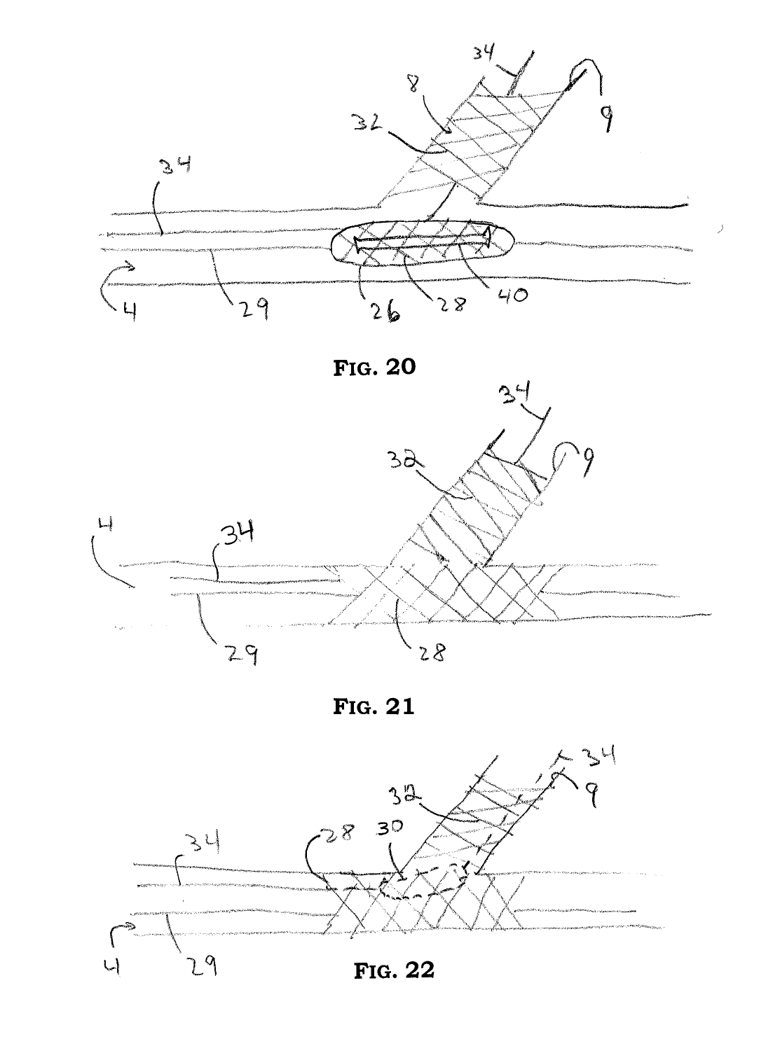

One exemplary embodiment of a method of deploying a stent assembly 20 of the present disclosure for stenting of both a side branch lumen 8 and main vessel lumen 4 of a bifurcated lumen 2 (FIGS. 13A and 13B show the stent assembly 20 already in position) is described as follows. In this exemplary method the side branch stent unit 24 is deployed before the main branch stent unit 22. A bifurcated stent assembly 20 is provided according to any of the embodiments as described hereinabove. FIG. 14 shows a main branch lumen 4 and a side branch lumen 8 with main branch and side branch guide wires 29 and 34 in place in the lumens. The side branch stent unit 24 is inserted in the side branch lumen 8 (FIG. 15). The main branch balloon 26 is inserted in the main lumen 4 and the balloon 26 is then expanded (FIGS. 16-17). The operator pulls back on the side branch vessel guide wire 34 until resistance is felt. The side branch balloon 30 is then expanded so that the side branch stent 32 is expanded against the side branch lumen inner wall 9 (FIGS. 17-18). The main branch balloon 26 is then deflated. The side branch stent balloon 30 is removed and then the main branch balloon 26 is removed (FIG. 19). The main branch stent unit 22 is then inserted over both the main branch guide wire 29 and the side branch guide wire 34 into the main lumen 4 (FIG. 20) and advanced and the main branch stent unit 22 is deployed in the main lumen 4 (FIG. 21). While the main branch balloon 26 and main branch stent 28 remain in position the side branch balloon 30 is inserted over the side branch guide wire 34 outside of the body so that the side branch balloon 30 can be easily positioned inside both the side branch stent 32 and straddle into the main branch stent 28. The side branch balloon 30 is advanced over the side branch guide wire 34 (FIG. 22) in the body and the main branch balloon 26 and side branch balloon 30 are inflated (FIG. 23).

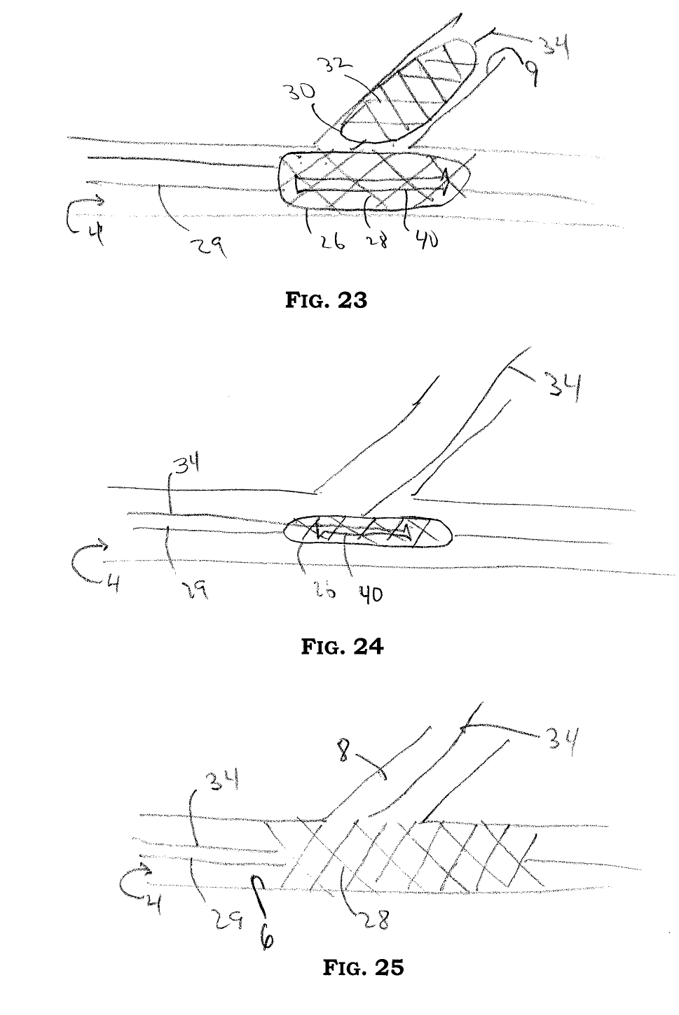

A second exemplary embodiment of a method of deploying a stent assembly 20 according to the present disclosure for provisional stenting of a bifurcated lumen 2 comprises the following. A bifurcated stent assembly 20 is provided in any of the apparatus embodiments as described hereinabove. The main branch stent unit 22 is inserted over both the main branch guide wire 29 and the side branch guide wire 34 and the side branch guide wire 34 is inserted into the sheath 40. This assembly 22 is then inserted into the main branch lumen 4 (FIG. 24) and is advanced to the desired implantation site. The main branch balloon 26 is expanded and the main branch stent 28 is expanded against the main branch lumen inner wall 6.

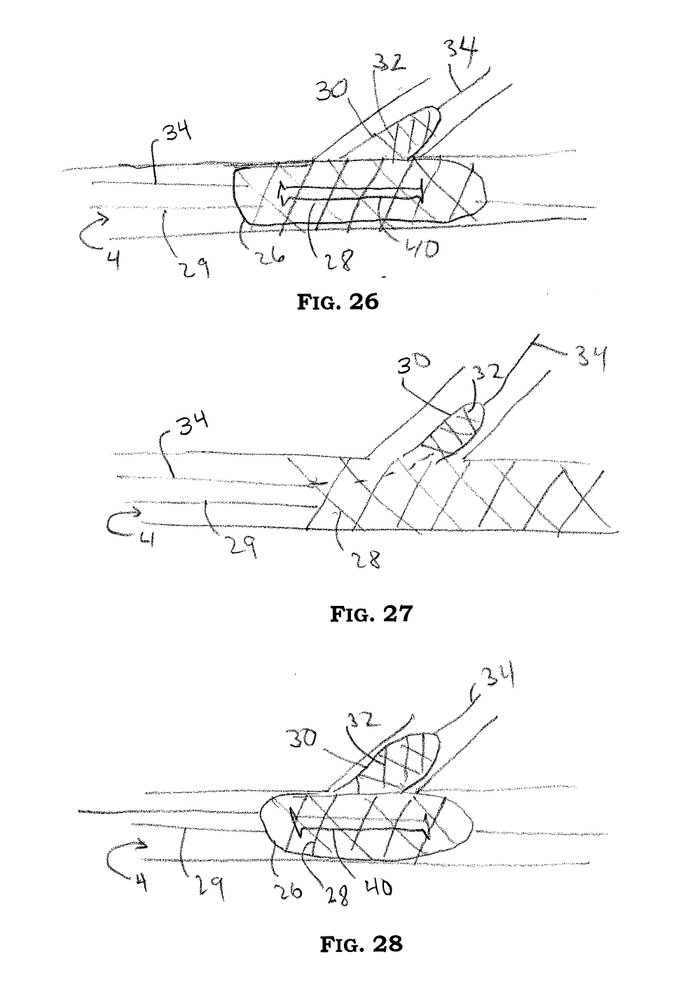

If no stenting of the side branch lumen 8 is required then the side branch balloon 30 is advanced over the side branch guide wire 34 (FIG. 25) and the main branch balloon 26 and the side branch balloon 30 are inflated (FIG. 26).

If stenting of the side branch lumen 8 is required, then the side branch stent unit 24 is advanced over the side branch guide wire 34 and positioned in the side branch lumen 8. The main branch balloon 26 is then inflated. The user pulls back on the side branch guide wire 34 until resistance is felt. The side branch stent 28 is then deployed (FIG. 27). The main branch balloon 26 is then deflated and the side branch balloon 30 is retracted slightly. The main branch balloon 26 and the side branch balloon 30 are inflated (FIG. 28).

Expansion of the main branch balloon 26 causes expansion of the sides of the sheath 40, thereby causing separation, parting, rupture, or the like of the frangible portion of the sheath (FIGS. 11-12). The distance between the top edge 52 and bottom edge 54 of the uninflated main branch balloon 26 is shown as distance D₁ in FIG. 11. When the balloon inflates, the distance between the top edge 52 and bottom edge 54 increases to distance D₂ (FIG. 12). In the alternative embodiment of the sheath 40 having the spaced alternating hooks 76 shown in FIG. 8, the separation upon expansion of the main branch balloon 26 causes the hooks 76 facing one way to separate or angle away from the hooks 77 facing the opposite way, thus releasing the guide wire from the hooks.

Upon insertion and expansion of both the main vessel stent and the branch vessel stent, the configuration may be as shown in FIG. 13.

A feature of the stent assembly 20 of the present disclosure is that both the main branch balloon 26 and the side branch balloon 30 can be inflated simultaneously, thereby avoiding the problem of crushing one stent while expanding the other.

The present invention also provides a kit comprising a stent assembly 20 (including a main branch stent unit 22 and a side branch stent unit 24 as described hereinabove) plus guide wires 29 and 34. The kit may also include at least one catheter, a syringe, and one or more shafts over which the balloons may pass. The stent is mounted on the balloon and the balloon is slide over the shaft. The shaft is hollow and the guide wire passes through the shaft lumen, as is known to those skilled in the art.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims.

It should further be noted that any patents, applications and publications referred to herein are incorporated by reference in their entirety.

What is claimed is:

1. A bifurcated stent assembly, comprising:
a) a main branch stent unit comprising
i) a main branch tubular stent,
ii) a main branch expandable member at least partially disposed within said stent material and having
a) an outer wall,
b) an inner wall,
c) a proximal end,
d) a distal end,
e) an expandable portion disposed between said proximal and said distal ends, and
f) a sheath associated with said outer wall of said expandable portion and being sized to allow a guide wire to pass therethrough, said sheath comprising
(1) an inner surface and an outer surface,
(2) a proximal opening,
(3) a distal opening,
(4) a top portion associated with said outer wall of said expandable member,
(5) a bottom portion generally parallel to and proximate to said top portion and associated with said outer wall of said expandable member, and,
(6) a frangible portion disposed between said proximal and distal openings and between said top and bottom portions, said frangible portion having a closed first configuration having a generally tubular shape when said main branch expandable member is in an uninflated configuration and an open second configuration when said main branch expandable member is in an inflated configuration whereby said frangible portion is separated and said top and bottom portions are separated so as to define a sheath edge opening therethrough, allowing a guide wire which is disposed within said sheath to be removable from said sheath via said sheath edge opening; and,
b) a side branch stent unit comprising
i) a side branch tubular stent,
ii) a side branch expandable member at least partially disposed within said stent material and having
a) a proximal end,
b) a distal end, and,
c) an expandable portion disposed between said proximal and said distal ends.

2. The bifurcated stent assembly of claim 1, further comprising a main branch guide wire.

3. The bifurcated stent assembly of claim 1, further comprising a side branch guide wire.

4. The bifurcated stent assembly of claim 1, wherein said frangible portion comprises a top portion and a bottom portion joined together and having a series of perforations extending at least partially through said sheath wall outer surface.

5. The bifurcated stent assembly of claim 1, wherein said frangible portion comprises a top portion and a bottom portion joined together and having a reduced wall thickness.

6. The bifurcated stent assembly of claim 1, wherein said frangible portion comprises a top portion having a generally zigzag edge and a bottom portion having a generally zigzag edge which mates with and is proximate to said top portion edge.

7. The bifurcated stent assembly of claim 1, wherein said sheath top portion has a generally straight edge and said sheath bottom portion has a generally straight edge, said top portion edge and said bottom portion edge overlapping to define a frangible portion whereby said top portion edge and said bottom portion edge can separate and not overlap when said sheath is in said second open configuration.

8. The bifurcated stent assembly of claim 1, wherein said sheath top portion has an edge including at least one protruding portion alternating with at least one recessed portion and said sheath bottom portion has an edge including at least one protruding portion alternating with at least one recessed portion such that said top portion edge mates with and is proximate to said bottom portion edge, whereby when in said closed first configuration said sheath and said top and bottom portion edges form a generally tubular configuration and whereby when in said open second configuration said top portion edge and said bottom portion edge protruding and recessed portions separate.

9. The bifurcated stent assembly of claim 1, wherein said sheath comprises a plurality of loops of material, each loop having a top end and a bottom end associated with said outer wall of said main branch expandable member.

10. The sheath of claim 9, whereby each loop has a thickness and a portion of reduced thickness, said reduced thickness portion being capable of separating.

11. The bifurcated stent assembly of claim 1, wherein said sheath comprises a plurality of hooks of material having a degree of flexion, each hook having a base end associated with and extending generally orthogonally from said outer wall of said main branch expandable member.

12. The sheath of claim 11, whereby said plurality of hooks are disposed such that said bases of said hooks are alternately above and below an imaginary line parallel to an axis of said main branch expandable member such that a guide wire passing through said plurality of hooks can be removed from said sheath when said sheath is in said open second configuration.

13. A bifurcated stent assembly kit, comprising:
c) a main branch guide wire;
d) a side branch guide wire;
e) at least one guide catheter; and,
f) a stent assembly comprising
i) a main branch stent unit comprising
a) a main branch tubular stent,
b) a main branch expandable member at least partially disposed within said stent material and having
(1) an outer wall,
(2) an inner wall,
(3) a proximal end,
(4) a distal end,
(5) an expandable portion disposed between said proximal and said distal ends, and
(6) a sheath associated with said outer wall of said expandable portion and being sized to allow a guide wire to pass therethrough, said sheath comprising
(a) an inner surface and an outer surface,
(b) a proximal opening,
(c) a distal opening,
(d) a top portion associated with said outer wall of said expandable member,
(e) a bottom portion generally parallel to and proximate to said top portion and associated with said outer wall of said expandable member, and,
(f) a frangible portion disposed between said proximal and distal openings and between said top and bottom portions, said frangible portion having a closed first configuration having a generally tubular shape when said main branch expandable member is in an uninflated configuration and an open second configuration when said main branch expandable member is in an inflated configuration whereby said frangible portion is separated and said top and bottom portions are separated so as to define a sheath edge opening therethrough, allowing a guide wire which is disposed within said sheath to be removable from said sheath via said sheath edge opening; and,
ii) a side branch stent unit comprising
a) a side branch tubular stent,
b) a side branch expandable member at least partially disposed within said stent material and having
(1) a proximal end,
(2) a distal end, and,
(3) an expandable portion disposed between said proximal and said distal ends.

14. A balloon for a stent delivery system allowing for separation of a guide wire associated with said balloon from said balloon, comprising:
a) an expandable member having
i) an outer wall,
ii) an inner wall,
iii) a proximal end, and
iv) a distal end; and,
b) a sheath associated with said outer wall of said expandable member, said sheath comprising
i) an inner surface and an outer surface,
ii) a proximal opening,
iii) a distal opening,
iv) a top portion associated with said outer wall of said expandable member,
v) a bottom portion generally parallel to and proximate to said top portion and associated with said outer wall of said expandable member, and
vi) a frangible portion disposed between said proximal and distal openings and between said top and bottom portions, said frangible portion having a closed first configuration having a generally tubular shape when said expandable member is in an uninflated configuration and an open second configuration when said expandable member is in an inflated configuration whereby said frangible portion is separated and said top and bottom portions are separated so as to define a sheath edge opening therethrough,
whereby a wire which is disposed at least partially within said sheath may be from said sheath via said sheath edge opening when said expandable member is in said second inflated configuration.

15. A method for treating a bifurcated vessel having a main branch and a side branch, comprising:
a) providing a stent assembly comprising
iii) a main branch stent unit comprising
a) a main branch tubular stent,
b) a main branch expandable member at least partially disposed within said stent material and having
(1) an outer wall,
(2) an inner wall,
(3) a proximal end,
(4) a distal end,
(5) an expandable portion disposed between said proximal and said distal ends, and
(6) a sheath associated with said outer wall of said expandable portion and being sized to allow a guide wire to pass therethrough, said sheath comprising
(a) an inner surface and an outer surface,
(b) a proximal opening,
(c) a distal opening,
(d) a top portion associated with said outer wall of said expandable member,
(e) a bottom portion generally parallel to and proximate to said top portion and associated with said outer wall of said expandable member, and,
(f) a frangible portion disposed between said proximal and distal openings and between said top and bottom portions, said frangible portion having a closed first configuration having a generally tubular shape when said main branch expandable member is in an uninflated configuration and an open second configuration when said main branch expandable member is in an inflated configuration whereby said frangible portion is separated and said top and bottom portions are separated so as to define a sheath edge opening therethrough, allowing a guide wire which is disposed within said sheath to be removable from said sheath via said sheath edge opening; and,
iv) a side branch stent unit comprising
a) a side branch tubular stent,
b) a side branch expandable member at least partially disposed within said stent material and having
(1) a proximal end,
(2) a distal end, and,
(3) an expandable portion disposed between said proximal and said distal ends;
g) inserting the branch stent in said branch vessel;
h) inserting said main vessel balloon in said main vessel;
i) inflating said main vessel balloon;
j) pulling back on said branch vessel stent until resistance is felt;
k) deploying said branch vessel stent;
l) removing said branch vessel stent balloon;
m) inflating said main vessel balloon;
n) removing said branch vessel stent;
o) inserting said stent assembly over both said main vessel guide wire and said branch vessel guide wire;
p) inserting said stent assembly of step j) into said main vessel;
q) advancing and deploying said stent assembly in said main vessel;
r) advancing said branch vessel balloon over said branch vessel guide wire; and,
s) inflating said main vessel balloon and said branch vessel balloon.

16. A method for treating a bifurcated vessel having a main branch and a side branch, comprising:
b) providing a stent assembly comprising
i) a main branch stent unit comprising
a) a main branch tubular stent,
b) a main branch expandable member at least partially disposed within said stent material and having
(1) an outer wall,
(2) an inner wall,
(3) a proximal end,
(4) a distal end,
(5) an expandable portion disposed between said proximal and said distal ends, and
(6) a sheath associated with said outer wall of said expandable portion and being sized to allow a guide wire to pass therethrough, said sheath comprising
(a) an inner surface and an outer surface,
(b) a proximal opening,
(c) a distal opening,
(d) a top portion associated with said outer wall of said expandable member,
(e) a bottom portion generally parallel to and proximate to said top portion and associated with said outer wall of said expandable member, and,
(f) a frangible portion disposed between said proximal and distal openings and between said top and bottom portions, said frangible portion having a closed first configuration having a generally tubular shape when said main branch expandable member is in an uninflated configuration and an open second configuration when said main branch expandable member is in an inflated configuration whereby said frangible portion is separated and said top and bottom portions are separated so as to define a sheath edge opening therethrough, allowing a guide wire which is disposed within said sheath to be removable from said sheath via said sheath edge opening; and,
ii) a side branch stent unit comprising
a) a side branch tubular stent,
b) a side branch expandable member at least partially disposed within said stent material and having
(1) a proximal end,
(2) a distal end, and,
(3) an expandable portion disposed between said proximal and said distal ends;
a) providing a main branch guide wire and a side branch guide wire;
b) inserting said main branch stent over said main branch guide wire and said side branch guide wire;
c) inserting said stent assembly into a main branch lumen and advancing said stent assembly to a site for implantation;
d) inflating said main branch expandable member and expanding said main branch stent;
e) advancing said side branch stent unit over said side branch guide wire into a side branch lumen;
f) inflating said main branch expandable member;
g) pulling on said side branch guide wire until resistance is felt;
h) inflating said side branch expandable member and expanding said side branch stent;
i) deflating said main branch expandable member;
j) retracting at least partially said side branch expandable member;
k) inflating said main branch expandable member and said side branch expandable member,
whereby initial expansion of said main branch expandable member causes said frangible portion of said sheath to come apart such that said side branch guide wire can be released from said sheath.
